(19) United States
(12) Patent Application Publication
(10) Pub. No.: US 2025/0295667 A1
(43) Pub. Date: Sep. 25, 2025

(54) PI3K-ALPHA INHIBITOR AND MTOR INHIBITOR COMBINATIONS FOR CANCER TREATMENT

(71) Applicant: Certis Oncology Solutions, Inc., San Diego, CA (US)

(72) Inventors: Long Hoang Do, Solana Beach, CA (US); Yuan Hung Chien, San Diego, CA (US); Raffaella Pippa, Del Mar, CA (US); Peter Ellman, San Diego, CA (US)

(21) Appl. No.: 19/226,065

(22) Filed: Jun. 2, 2025

Related U.S. Application Data
(62) Division of application No. 18/614,442, filed on Mar. 22, 2024.

(51) Int. Cl. A61K 31/5377; A61K 31/436; A61K 31/4439; A61K 31/501; A61K 31/519; A61K 31/553; A61K 31/675; A61P 35/00

(52) U.S. Cl. CPC A61K 31/5377; A61K 31/436; A61K 31/4439; A61K 31/501; A61K 31/519; A61K 31/553; A61K 31/675; A61P 35/00

(57) ABSTRACT

The present invention relates to compositions comprising a combination of PI3K-ALPHA and MTOR inhibitors and methods of combination therapy for administering to subjects in need thereof for the treatment of solid tumor cancers.

### CROSS-REFERENCE TO RELATED APPLICATION(S)

This application is a Divisional application Ser. No. 18/614,442, filed Mar. 22, 2024, the disclosure of which is hereby incorporated by reference in its entirety.

### BACKGROUND

The present disclosure relates to combination-therapy treatments for cancer.

### SUMMARY OF DISCLOSURE

According to one aspect of the present disclosure, a method of treating or ameliorating the effects of a solid tumor cancer in a subject in need thereof is provided.

In some embodiments, the method comprises: administering to the subject an effective amount of a first anti-cancer agent, which is a PI3K-alpha inhibitor or a pharmaceutically acceptable salt thereof. The method also comprises administering to the subject an effective amount of a second anti-cancer agent, which is a mTOR inhibitor or a pharmaceutically acceptable salt thereof. The administration of the first and second anti-cancer agents are to treat or ameliorate the effects of a solid tumor cancer.

In a further aspect of the present disclosure, a pharmaceutical composition for treating or ameliorating the effects of a solid tumor cancer in a subject in need thereof is provided. The pharmaceutical composition comprises a pharmaceutically acceptable diluent or carrier. The pharmaceutical composition also comprises an effective amount of (i) a first anti-cancer agent, which is a PI3K-alpha inhibitor or a pharmaceutically acceptable salt thereof, and (ii) a second anti-cancer agent, which is an mTOR inhibitor or a pharmaceutically acceptable salt thereof. Additionally, the administration of the first and second anti-cancer agents provides a synergistic effect compared to administration of either anti-cancer agent alone.

In some embodiments of the disclosed method, the PI3K-alpha inhibitor is selected from the group consisting of: Alpelisib, Omipalisib, Gedatolisib, Inavolisib, PF-04691502, and Serabelisib.

In some embodiments of the disclosed method, the mTOR inhibitor is selected from the group consisting of: Everolimus, AXD-8055, Tacrolimus, and Ridaforolimus.

In some embodiments, the PI3K-alpha inhibitor is Alpelisib and the mTOR inhibitor is Everolimus.

In some embodiments, the PI3K-alpha inhibitor is Alpelisib and the mTOR inhibitor is AXD-8055.

In some embodiments, the PI3K-alpha inhibitor is Alpelisib and the mTOR inhibitor is Tacrolimus.

In some embodiments, the PI3K-alpha inhibitor is Alpelisib and the mTOR inhibitor is Ridaforolimus.

In some embodiments, the PI3K-alpha inhibitor is Omipalisib and the mTOR inhibitor is Everolimus.

In some embodiments, the PI3K-alpha inhibitor is Omipalisib and the mTOR inhibitor is AXD-8055.

In some embodiments, the PI3K-alpha inhibitor is Omipalisib and the mTOR inhibitor is Tacrolimus.

In some embodiments, the PI3K-alpha inhibitor is Omipalisib and the mTOR inhibitor is Ridaforolimus.

In some embodiments, the PI3K-alpha inhibitor is Gedatolisib and the mTOR inhibitor is Everolimus.

In some embodiments, the PI3K-alpha inhibitor is Gedatolisib and the mTOR inhibitor is AXD-8055.

In some embodiments, the PI3K-alpha inhibitor is Gedatolisib and the mTOR inhibitor is Tacrolimus.

In some embodiments, the PI3K-alpha inhibitor is Gedatolisib and the mTOR inhibitor is Ridaforolimus.

In some embodiments, the PI3K-alpha inhibitor is Inavolisib and the mTOR inhibitor is Everolimus.

In some embodiments, the PI3K-alpha inhibitor is Inavolisib and the mTOR inhibitor is AXD-8055.

In some embodiments, the PI3K-alpha inhibitor is Inavolisib and the mTOR inhibitor is Tacrolimus.

In some embodiments, the PI3K-alpha inhibitor is Inavolisib and the mTOR inhibitor is Ridaforolimus.

In some embodiments, the PI3K-alpha inhibitor is PF-04691502 and the mTOR inhibitor is Everolimus.

In some embodiments, the PI3K-alpha inhibitor is PF-04691502 and the mTOR inhibitor is AXD-8055.

In some embodiments, the PI3K-alpha inhibitor is PF-04691502 and the mTOR inhibitor is Tacrolimus.

In some embodiments, the PI3K-alpha inhibitor is PF-04691502 and the mTOR inhibitor is Ridaforolimus.

In some embodiments, the PI3K-alpha inhibitor is Serabelisib and the mTOR inhibitor is Everolimus.

In some embodiments, the PI3K-alpha inhibitor is Serabelisib and the mTOR inhibitor is AXD-8055.

In some embodiments, the PI3K-alpha inhibitor is Serabelisib and the mTOR inhibitor is Tacrolimus.

In some embodiments, the PI3K-alpha inhibitor is Serabelisib and the mTOR inhibitor is Ridaforolimus.

In some embodiments, the subject is a mammal.

In some embodiments, the mammal is selected from the group consisting of: humans, primates, mice, farm animals, and domestic animals.

In some embodiments, the mammal is a human.

In some embodiments, the administration of the first and second anti-cancer agents provides a synergistic effect compared to the administration of either anti-cancer agent alone.

In some embodiments, the synergistic effect is assessed at least in part using a ZIP synergy assessment.

In some embodiments, the synergistic effect is assessed at least in part using a Bliss synergy assessment.

In some embodiments, the synergistic effect is assessed at least in part using a HSA synergy assessment.

In some embodiments, the synergistic effect is assessed at least in part using a Loewe synergy assessment.

In some embodiments, the synergistic effect is assessed by taking a maximal synergy among a plurality of synergy assessment scores.

In some embodiments, the synergistic effect is assessed by taking an average of a plurality of synergy assessment scores.

In some embodiments, one or more of the plurality of synergy assessment scores are selected from the group consisting of a ZIP synergy score, a Bliss synergy score, a Loewe synergy score, and an HSA synergy score.

In some embodiments, the subject has a 10% lower rate of tumor growth 10 days after administration of the effective amount of the first and second anti-cancer agents as compared to a control.

In some embodiments, the subject has a 20% lower rate of tumor growth 10 days after administration of the effective amount of the first and second anti-cancer agents as compared to a control.

In some embodiments, the subject has a 50% lower rate of tumor growth 10 days after administration of the effective amount of the first and second anti-cancer agents as compared to a control.

In some embodiments, the subject has a 10% lower rate of tumor growth 20 days after administration of the effective amount of the first and second anti-cancer agents as compared to a control.

In some embodiments, the subject has a 20% lower rate of tumor growth 20 days after administration of the effective amount of the first and second anti-cancer agents as compared to a control.

In some embodiments, the subject has a 50% lower rate of tumor growth 20 days after administration of the effective amount of the first and second anti-cancer agents as compared to a control.

In some embodiments, the subject has a 10% lower rate of tumor growth 24 days after administration of the effective amount of the first and second anti-cancer agents as compared to a control.

In some embodiments, the subject has a 20% lower rate of tumor growth 24 days after administration of the effective amount of the first and second anti-cancer agents as compared to a control.

In some embodiments, the subject has a 50% lower rate of tumor growth 24 days after administration of the effective amount of the first and second anti-cancer agents as compared to a control.

In some embodiments, an additional administration of at least one additional therapeutic agent is provided, where the at least one additional therapeutic agent is selected from the group consisting of: alkylating agents, antibiotics, cytotoxic antibiotics, antimetabolites, biologic response modifiers, histone deacetylase inhibitors, hormonal agents, monoclonal antibodies, protein kinase inhibitors, taxanes, topoisomerase inhibitors, vinca alkaloids, photoactive therapeutic agents, anti-angiogenesis agents, radiosensitizing agents, radionuclides, immunomodulators, cytotoxic agents, and combinations thereof.

In some embodiments, a pharmaceutical composition is in a unit dosage form comprising both the first and second anti-cancer agents.

In some embodiments, the pharmaceutical composition comprises a first anti-cancer agent in a first unit dosage form and a second anti-cancer agent in a second unit dosage form, wherein the first unit dosage form is separate from the second unit dosage form. In some embodiments, the first unit dosage form is less than the second unit dosage form. In some embodiments, the first unit dosage form is substantially the same as the second unit dosage form. In some embodiments, the first unit dosage form is the same as the second unit dosage form. In some embodiments, the first unit dosage form is greater than the second unit dosage form.

In some embodiments, the first and second anti-cancer agents are co-administered to the subject.

In some embodiments, the first and second anti-cancer agents are co-administered to the subject serially. In some embodiments, the first anti-cancer agent is administered to the subject before the second anti-cancer agent. In some embodiments, the second anti-cancer agent is administered to the subject before the first anti-cancer agent.

In some embodiments, a data structure comprising a decision tree random forest model is utilized to uncover biomarkers to predict the efficacy of a PI3K-alpha inhibitor or a pharmaceutically acceptable salt thereof and a second anti-cancer agent, wherein the second anti-cancer agent is an mTOR inhibitor or a pharmaceutically acceptable salt thereof.

In some embodiments, a method of predicting the response to treating or ameliorating the effects of a solid tumor cancer using measurements of gene expression biomarkers of one or more genes selected from the group consisting of: RPS16, HERC6, L2HGDH, AAGAB, CENPN, CHMP2B, and TMED10.

In some embodiments, a method of predicting the response to treating or ameliorating the effects of a solid tumor cancer further uses assessments of the ratio of predictive biomarkers of the one or more gene expression biomarker measurements as compared to a geometric mean of measurements of one or more housekeeping genes selected from the group consisting of: RPL27, RPL4, UBA52, HNRNPK, RPL30.

In some embodiments, a method of predicting the response to treating or ameliorating the effects of a solid tumor cancer using a molecular description of a PI3K-alpha inhibitor or a pharmaceutically acceptable salt thereof.

In some embodiments, a method of predicting the response to treating or ameliorating the effects of a solid tumor cancer using a molecular description of an mTOR inhibitor or a pharmaceutically acceptable salt thereof.

In some embodiments, a method of predicting the response to treating or ameliorating the effects of a solid tumor cancer using a molecular fingerprint of a PI3K-alpha inhibitor or a pharmaceutically acceptable salt thereof.

In some embodiments, a method of predicting the response to treating or ameliorating the effects of a solid tumor cancer using a molecular fingerprint of an mTOR inhibitor or a pharmaceutically acceptable salt thereof.

### DESCRIPTION OF THE FIGURES

The objects and features of the invention can be better understood with reference to the following detailed description and accompanying drawings.

FIG. 1 provides an illustration of a plurality of PI3K-alpha inhibitor molecules.

FIG. 2 provides an illustration of a plurality of mTOR inhibitor molecules.

FIG. 3 illustrates in-vivo pharmacology test results of a Patient Derived Xenograft (PDX) lung cancer model, CRT00295. On the x-axis, tumor volume is represented, and on the y-axis, study days is represented. Further, two sets of data are plotted: a first set of data provides a PDX model of a mouse that is a vehicle control, while a second set of data provides a PDX model of a mouse treated with a combination of Everolimus and Alpelisib.

FIG. 4 illustrates in-vivo pharmacology test results of a Patient Derived Xenograft (PDX) gastric cancer model, CRT00292. On the x-axis, tumor volume is represented, and on the y-axis, study days is represented. Further, two sets of data are plotted: a first set of data provides a PDX model of a mouse that is a vehicle control, while a second set of data provides a PDX model of a mouse treated with a combination of Everolimus and Alpelisib.

FIG. 5 illustrates in-vivo pharmacology test results of a Patient Derived Xenograft (PDX) pancreatic cancer model, CRT00559. On the x-axis, tumor volume is represented, and on the y-axis, study days is represented. Further, two sets of data are plotted: a first set of data provides a PDX model of a mouse that is a vehicle control, while a second set of data provides a PDX model of a mouse treated with a combination of Everolimus and Alpelisib.

FIG. 6 illustrates a comprehensive correlation analysis between predicted tumor growth inhibition (TGI) vs. actual TGI from in-vivo Everoliumus and Alpelisib combination pharmacology studies across 11 PDX solid tumor models (Table 4) showing a positive Pearson's correlation of r=0.63.

### DETAILED DESCRIPTION

The present disclosure relates to compositions comprising inhibitors of PI3K-Alpha and MTOR for the treatment of solid tumors with activating RAS mutations.

The PI3K and mTOR signaling pathways are one of the most frequently dysregulated pathways in human cancers. While there are multiple drugs in these categories that are available to separately modulate dysregulated targets along these individual pathways, there may be synergistic effects in treating patients with a combination of these drugs that affect a distinct target in each pathway. As such, it is beneficial to compare benefits of a combination of drugs to identify which drugs may have synergistic effects against the progression of a particular lung cancer of a particular patient. Further, by identifying drug combinations that are synergistic against cancers of an individual patient, overarching drug therapies may also be developed that are effective across multiple patients having related cancers.

Recent advances in patient-derived xenograft (PDX) modeling technology has allowed for anticipated patient response to be assessed against a variety of treatment options. In particular, PDX models allow tumor tissues, or other cancer-related tissues, to be implanted into an immunocompromised or humanized vessel. Examples of vessels that are used in PDX models include mice. By assessing combination drugs across varying dosage amounts and against a patient's own cancer tissue, tailored drug recommendations can be applied to benefit patients who are in need of cancer treatment.

The present disclosure recognizes the benefits of providing multiple PDX models for assessing a patient's response to various combinations of PI3K-alpha and mTOR inhibitors. The present disclosure also recognizes the benefits of providing further PDX models for assessing varying dosages of drugs across the various combinations of PI3K-alpha and mTOR inhibitors.

The present disclosure also recognizes the benefits of using in vitro models for assessing the effectiveness of anti-cancer agents against biological material derived from a subject. In some embodiments, the subject is a patient that has lung cancer. In some embodiments, the subject is a vehicle control.

### Definitions

As used herein, the following terms have the meanings ascribed to them unless specified otherwise.

As used in the specification and claims, the singular form “a”, “an” and “the” include plural references unless the context clearly dictates otherwise. For example, the term “a cell” includes a plurality of cells, including mixtures thereof.

Also, as used in the description herein and throughout the claims that follow, the meaning of “in” includes “in” and “on” unless the context clearly dictates otherwise.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

“Data set” refers to a set of data whose elements are data points.

“Data point” refers to an element of a data set, e.g., a subject sample, identified for example, by a label or patient number identifying the source of the sample.

As used herein, the term “correspond/correspondence to” refers to the ability of a system or component of a system to receive input data from another system or component of a system and to provide an output response in response to the input data. “Output” may be in the form of data or may be in the form of an action taken by the system or component of the system.

As used herein, a “computer program product” refers to the expression of an organized set of instructions in the form of natural or programming language statements that is contained on a physical media of any nature (e.g., written, electronic, magnetic, optical or otherwise) and that may be used with a computer or other automated data processing system of any nature (but preferably based on digital technology). Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in preselected ways may be provided in a number of forms, including, but not limited to, original source code, assembly code, object code, machine language, encrypted or compressed versions of the foregoing and any and all equivalents.

### In Vitro/Ex-Vivo Anti-Cancer Agent Response

The present disclosure provides methods of assessing various drug combinations against biological material derived from a subject through the use of in vitro platforms. These in vitro platforms may include tumor models that may be based on portions of a biological sample of a patient that has lung cancer. In some embodiments, a biological sample may comprise at least a portion of a tumor of a patient having lung cancer. In some embodiments, these in vitro platforms may be based on a cell line that has been developed based on a cell line of a patient having lung cancer.

When assessing a patient's response to a particular drug combination, biological material that corresponds to the patient may be exposed to many different dosage combinations of the particular drug combination. The use of in vitro methods allows for multiple tests to be run against biological material that corresponds to the patient. In some embodiments, biological material that corresponds to a patient may be tested against drug dosages of a first anti-cancer agent and a second anti-cancer agent within the drug combination in a checkerboard assay. In some embodiments, the dosages for the first anti-cancer agent may range from 0.000095 uM to 100 uM. In some embodiments, a range of dosages of the first anti-cancer agent may include 0.000095 uM, 0.00038 uM, 0.0015 uM, 0.0061 uM, 0.024 uM, 0.098 uM, 0.39 uM, 1.56 uM, 6.25 uM, 25 uM, and 100 uM. In some embodiments, the dosages for the second anti-cancer agent may also range from 0.000095 uM to 100 uM. In some embodiments, the dosages for the second anti-cancer agent may also include include 0.000095 uM, 0.00038 uM, 0.0015 uM, 0.0061 uM, 0.024 uM, 0.098 uM, 0.39 uM, 1.56 uM, 6.25 uM, 25 uM, and 100 uM.

In some other embodiments, the range of dosages may differ between the first anti-cancer agent and the second anti-cancer agent. In some embodiment, the range of dosages may be medication-specific. For example, if there is an anti-cancer agent that has high volatility over a smaller dosage range, then the range of the dosages for that anti-cancer agent may be smaller as compared to another anti-cancer agent having high volatility over a larger dosage range.

In some embodiments, each instance a dosage for the first anti-cancer agent may be assessed in a drug combination for each dosage for the second anti-cancer agent. As such, in some embodiments, the biological material that corresponds to a patient having cancer may be assessed in 11 separate drug combinations where a first anti-cancer agent may have a dosage of 0.000095 uM, and where a second anti-cancer agent may have a dosage of 0.000095 uM; 0.00038 uM; 0.0015 uM; 0.0061 uM; 0.024 uM; 0.098 uM; 0.39 uM; 1.56 uM; 6.25 uM; 25 uM; and 100 uM, respectively. In this way, 11×11, or 121, drug combinations may be assessed for a first anti-cancer agent having dosages comprising 0.000095 uM, 0.00038 uM, 0.0015 uM, 0.0061 uM, 0.024 uM, 0.098 uM, 0.39 uM, 1.56 uM, 6.25 uM, 25 uM, and 100 uM, and a second anti-cancer agent having dosages comprising 0.000095 uM, 0.00038 uM, 0.0015 uM, 0.0061 uM, 0.024 uM, 0.098 uM, 0.39 uM, 1.56 uM, 6.25 uM, 25 uM, and 100 uM.

The CTG assay is a widely used method to assess pharmacological testing of a combination of two drugs using a checkerboard pattern in cancer cells. The checkerboard pattern is created by combining two drugs at different concentrations in a matrix format. The cells are then incubated with the drug combination for a specific period of time, and the cell viability is measured using a colorimetric assay. The results are analyzed to determine the synergistic, additive, or antagonistic effects of the drug combination. This method is useful in identifying the optimal drug combination and concentration for cancer treatment.

### Synergy Models

Once biological response data has been gathered from the in vitro assessments, the resulting data may be assessed for synergistic characteristics. Synergy measures a degree of interaction when two or more drugs are combined. In some cases, the drugs will act independently of one another; in some cases, the drugs will exhibit positive, synergistic effects that promote greater medicinal effects when working together than when working alone; and in some cases, the drugs will exhibit negative, antagonistic effects that show one or more drugs acting in a way that is less effective than if the drugs were administered alone.

In assessing synergy of a particular drug combination, observed drug combination responses may be compared against expected responses using a reference model that assumes no interaction between drugs. The reference models used for assessing this metric can vary, and are discussed below. In some embodiments, the calculations of synergistic assessment may be aided by using a computer program, such as SynergyFinder (v2).

In some embodiments, one or more reference models may be used in assessing synergy characteristics of drug combinations for treating a patient that has lung cancer. Four exemplary models include the Bliss independence model (hereinafter referred to as the “Bliss” model), the Loewe additivity model (hereinafter referred to as the “Loewe” model), the Zero interaction potency model (hereinafter referred to as the “ZIP” model), and the Highest Single Agent model (hereinafter referred to as the “HAS” model).

### Review of Synergy Reference Models

Synergy reference models can be used to help answer the question, “if there is no interaction between drugs, what would be the expected drug combination response when evaluating two anti-cancer agents?” While that question is straight-forward, the possible responses can vary depending on the underlying assumptions used in a given model.

The Bliss model, for example, provides for an expected drug combination response, y_{BLISS}, to be computed as the product of the individual drug responses, y₁ and y₂, of a first and second anti-cancer agent, respectively:

$${y}_{BLISS} = {y}_{1} + ( 1 - {y}_{1} ) {y}_{2} = {y}_{1} + {y}_{2} - {y}_{1} {y}_{2}$$

Of note, the Bliss model does not take into account the potential of dose-response relationships of individual drugs. This additional factor is, however, considered in the Loewe additivity model. Under Loewe, dose-response functions of a first anti-cancer agent and a second anti-cancer agent are represented as ƒ₁(x) and ƒ₂ (x), respectively.

In order to calculate the appropriate dose at which each drug alone produces the expected response y_{LOEWE}, an inverse function ƒ⁻¹ is calculated so as to map back the response, y, back to the dose, x. Additionally, the Loewe additivity model provides that y_{LOEWE} must satisfy:

$$[ {x}_{1} / ( {{f}_{1}}^{- 1} ( {y}_{LOEWE} ) ) ] + [ {x}_{2} / ( {{f}_{2}}^{- 1} ( {y}_{LOEWE} ) ) ] = 1$$

Under the HSA model, for assessing two drugs in a drug combination, the expected combination effect for multiple drugs is assessed as the cumulative maximum of the single-drug response for each component (e.g., y₁, y₂). As such, a synergy score is assessed by subtracting the cumulative maximum of the single-drug response from each component from the combined observed effect (e.g. y_{1,2}) of the multiple drugs taken in combination.

Using an example of three drugs 1, 2, 3 being combined, the synergy assessment under the HSA model would be:

$${y}_{HSA} = {y}_{1 , 2 , 3} - max ( {y}_{1} + {y}_{2} + {y}_{3} )$$

The ZIP model combines benefits of other reference models, namely the Bliss model and the Loewe model, and allows the expected response to incorporate factors for how the combination of drug components will affect the respective dose-response curves for each individual drug component.

In the zip model, for a drug “1”, y represents the drug effect; x represents the dosage; m represents the dose of a drug that produces the mid-point effect between the maximum and minimum effects of the drug; and lambda represents a shape parameter indicating the sigmoidicity, or slope, of a dose-response curve for the drug. As such, the ZIP model can be represented as:

$${y}_{ZIP} = \frac{{( \frac{{x}_{1}}{{m}_{1}} )}^{{λ}_{1}}}{1 + {( \frac{{x}_{1}}{{m}_{1}} )}^{{λ}_{1}}} + \frac{{( \frac{{x}_{2}}{{m}_{2}} )}^{{λ}_{2}}}{1 + {( \frac{{x}_{2}}{{m}_{2}} )}^{{λ}_{2}}} - \frac{{( \frac{{x}_{1}}{{m}_{1}} )}^{{λ}_{1}}}{1 + {( \frac{{x}_{1}}{{m}_{1}} )}^{{λ}_{1}}} \frac{{( \frac{{x}_{2}}{{m}_{2}} )}^{{λ}_{2}}}{1 + {( \frac{{x}_{2}}{{m}_{2}} )}^{{λ}_{2}}} .$$

### Use of Synergy Reference Models

Once one or more measures of synergy have been assessed for a particular drug combination, the data may be used to create a synergy score. A synergy score may represent an assessment from a single synergy assessment or from multiple synergy assessments. In some embodiments, a combined synergy score may represent an average of component synergy scores that have been combined. In some embodiments, a weighted combined synergy score may comprise multiple synergy components that are weighted to favor one or more assumptions of particular reference models.

In some embodiments, assessed synergy characteristics, such as synergy scores, may be used to determine treatment options for a patient having lung cancer. In some embodiments, other factors, such as a patient's tolerance for a particular drug or presence of drug allergies, may contribute to decisions for patient care in combination with synergy scores.

### Identification of the Optimal Drug Concentration of Each Combination Drug and Calculation of the Area Under the Dose Response Curve (AUC).

For every drug combination tested against a cancer cell line, a determination of the most optimal drug concentration for each of the drugs tested is determined by finding the synergy model containing the highest summary synergy score (max synergy) and then the concentration of the two drugs with the highest average synergy scores for each concentration.

The area under the dose response curve (AUC) is used to compare the efficacy of different combination treatments. In single drug experiments, the AUC is calculated by plotting the viability of cancer cells to different doses of a drug. To calculate the AUC for a drug combination, each drug in the combination is calculated separately by varying the dose of the first drug while keeping the dose of the second drug fixed at the optimal drug concentration found in [107]. The AUC calculation is repeated for the second drug and a final AUC for the combination is determined by averaging the results from the two separate AUC calculations.

### Machine Learning

The present disclosure uses the experimental results from the testing of multiple combinations of PI3K-Alpha and mTOR inhibitors to train at least one learning algorithm that is used in a prediction model, wherein the trained prediction model is capable of qualifying a plurality of drug combinations from the PI3K-Alpha and mTOR inhibitors in terms of their predicted biological response to a human cancer tissue exhibiting a specific set of predictive biomarkers.

In some embodiments of the disclosed method, at least one learning algorithm comprises a multivariate regression algorithm, including random-forest, support vector machines, and artificial neural networks.

In some embodiments of the disclosed method, at least one learning algorithm comprises a feature selection algorithm, preferably a Boruta algorithm.

### Data Elements

Data elements are features of a data point representing characteristics of the data point. For example, in one aspect, data elements represent expression values of a plurality of different genes in a sample from a patient having a disease shared in common among patients contributing samples to the data set. Any method for expression profiling known in the art may be used to obtain expression values and is encompassed within the scope of the invention.

Data elements (e.g., gene expression values) can be obtained by transcriptional profiling and/or by proteome profiling. Transcriptional profiling techniques include, but are not limited to: Sequencing by synthesis (SBS), Northern blots, qPCR and RT-PCR-based differential display methods, nuclease protection, representation different analysis (RDA), suppression subtractive hybridization (SSH), and enzymatic degrading subtraction (EDS), gene array profiling, cDNA fingerprinting, subtractive hybridization, serial analysis of gene expression, or SAGE, and the like. Proteome profiling techniques include, but are not limited to: two-hybrid analysis, fluorescence resonance energy transfer (MET), two-dimensional gel electrophoresis, mass spectrometry (e.g., laser desorption/ionization mass spectrometry), fluorescence (e.g. sandwich immunoassay), surface plasmon resonance, ellipsometry and atomic force microscopy.

Other types of biomolecules which are differentially expressed may be profiled to provide data elements. For example, carbohydrates such as lectins (e.g., such as glycans) have diverse expression patterns which can provide data values for data elements comprising a data point.

Preferred methods of expression profiling are high throughput and obtain data elements from greater than about ten, greater than about 50, greater than about 100, greater than about 200, or greater than about 500 samples in data set.

Preferably, a data element is represented as a vector of numerical values including a value representing the level of a sample component represented by a data element and at least one other characteristic of the sample component/data element, such as its name or descriptor.

### Qualifying Data Elements

In the next step, data elements obtained from an expression profiling method are qualified using any sort of multivariate analysis. In one method qualification involves using a pattern recognition process, such as a regression model.

By this method, data elements are identified with high confidence values (a selected difference from a null (randomized) distribution being accepted as statistically significant with minimal false discovery, e.g., FDR≤0.05) and which are expressed qualitatively in the same manner (overexpressed or under-expressed in both data sets). Outliers of high confidence are ranked from those showing the greatest difference in expression between a test data point and a reference data point (i.e., the most diagnostic) to those which show the least amount of difference (i.e., least diagnostic).

Thus, for example, gene expression or protein expression data from a collection of samples may yield expression data on over twenty thousand genes or proteins: Each is a data element and its measured expression level is a data element value. After subjecting a data set to the selected form of analysis, the ability of each gene or protein, based on its expression level, to classify a particular sample (data point) as cancerous or non-cancerous and if cancerous, belonging to which cancer type class (form of biological state class) is determined, or “qualified.” Each gene or protein might then be ranked from most discriminating to least discriminating.

### Using Learning Algorithm in Predictive Model

The data elements in the intersection subset can be used in multivariate models to generate multivariate regression algorithms. To construct multivariate predictive models, the data from the plurality of data sets are combined and randomly divided into a discovery training set and a test set.

The performance of the panel of potential biomarkers identified from re-sampling and cross-comparison and derived predicted models are evaluated on the test set to identify those biomarkers which survive and which remain highly diagnostic of at least one common characteristic. Predictive models are validated on independent data elements from one or more new data sets sharing at least one common characteristic and which have not been involved in biomarker discovery and the model construction process. Independent validation may be performed on data sets which comprise larger populations of data points or are analyzed using different method (e.g., with a different expression profiling technique from the one used to initially obtain the data elements, such as by an immunoassay), obtaining validation training sets that may be used to identify the most highly discriminatory biomarkers of those being tested. Statistical methods for evaluation of validation data sets include mean-square error, sensitivity and specificity estimation and receiver-operating characteristic (ROC) curve analysis, among others.

The multivariate regression algorithm thus generated can be tested against another independent “validation” data set to determine the ultimate power of the algorithm. The validation data set should be independent of all of the discovery data sets used to discover the biomarkers from which the regression algorithm was generated.

Biomarkers can be evaluated after re-sampling, though more preferably, after cross-comparison, to identify additional features of the biomarkers which can be used to characterize validation data sets. For example, sequence information for a peptide or nucleic acid biomarker may be determined. The additional feature(s) may be used to generate probes to test for the presence of the biomarker in test samples (new data points) in data sets used to validate the biomarker. Additional features may include sequence data regarding a larger sequence of which the biomarker sequence is a subsequence (e.g., sequence data for a gene or protein from which the nucleic acid or peptide was derived). Such data may be obtained by using the biomarker sequence to query a database, such as a gene sequence, protein sequence, or glycomic database. Using this method, the sequences of other markers can be identified if these markers are known in the databases.

Preferably, a data element is identified as a biomarker when it is able to predict with greater than 70%, preferably greater than 80%, and still more preferably, greater than 90% accuracy, the presence or absence of a characteristic of a member of a data set. In certain aspects, a plurality of data elements combined can provide the desired predictive value. In certain aspects, combinations with high predictive value may include data elements with lower confidence and may be more predictive than single data elements with higher confidence values. Combinations of data elements suitable for use as biomarkers may be identified by pairing in an ordered or random approach, for example.

### Validation by Xenograft Mouse Model

The assessment of drug combinations for a patient having lung cancer as discussed in the present disclosure may also benefit by using a xenograft mouse model. In some embodiments, within the xenograft mouse model, the biological material of the patient having cancer is implanted in parallel into multiple immune-deficient mice each subsequently treated in parallel with one of the plurality of drug combinations. Biological response of the treatment can then be assessed to determine effectiveness of the drug combination. In some embodiments of the disclosed method, the biological material of the patient having cancer is orthotopically implanted into multiple immune-deficient mice.

Patient derived xenografts (PDX) are models of cancer where the tissue or cells from a human patient's tumor are implanted into an immunodeficient or humanized mouse. PDX models are used to create an environment that allows for the natural growth of cancer, its monitoring, and corresponding treatment evaluations for the original patient as well as patients with similar cancer profiles.

### Tumor Xenotransplantation

Several types of immunodeficient mice can be used to establish PDX models: athymic nude mice, severely compromised immune deficient (SCID) mice, NOD-SCID mice, and recombination-activating gene 2 (Rag2)-knockout mice.[2] The mice used must be immunocompromised to prevent transplant rejection. The NOD-SCID mouse is considered more immunodeficient than the nude mouse, and therefore is more commonly used for PDX models because the NOD-SCID mouse does not produce natural killer cells.

When human tumors are resected, necrotic tissues are removed and the tumor can be mechanically sectioned into smaller fragments, chemically digested, or physically manipulated into a single-cell suspension. There are advantages and disadvantages in utilizing either discrete tumor fragments or single-cell suspensions. Tumor fragments retain cell-cell interactions as well as some tissue architecture of the original tumor, therefore mimicking the tumor microenvironment. Alternatively, a single-cell suspension enables scientists to collect an unbiased sampling of the whole tumor, eliminating spatially segregate subclones that are otherwise inadvertently selected during analysis or tumor passaging. However, single-cell suspensions subject surviving cells to harsh chemical or mechanical forces that may sensitize cells to anoikis, taking a toll on cell viability and engraftment success.

### Heterotopic and Orthotopic Implantation

Unlike creating xenograft mouse models using existing cancer cell lines, there are no intermediate in vitro processing steps before implanting tumor fragments into a murine host to create a PDX. The tumor fragments are either implanted heterotopically or orthotopically into an immunodeficient mouse. With heterotopic implantation, the tissue or cells are implanted into an area of the mouse unrelated to the original tumor site, generally subcutaneously or in subrenal capsular sites. The advantages of this method are the direct access for implantation, and ease of monitoring the tumor growth. With orthotopic implantation, scientists transplant the patient's tumor tissue or cells into the corresponding anatomical position in the mouse. Subcutaneous PDX models are unlikely to produce metastasis in mice, nor do they simulate the initial tumor microenvironment, with engraftment rates of 40-60%. Subrenal capsular PDX maintains the original tumor stroma as well as the equivalent host stroma and has an engraftment rate of 95%. Ultimately, it takes about 2 to 4 months for the tumor to engraft varying by tumor type, implant location, and strain of immunodeficient mice utilized; engraftment failure should not be declared until at least 6 months.[2] Researchers may use heterotopic implantation for the initial engraftment from the patient to the mouse, then use orthotopic implantation to implant the mouse-grown tumor into further generations of mice.

### Generations of Engraftments

The first generation of mice receiving the patient's tumor fragments are commonly denoted F0. When the tumor becomes sufficient in size in the F0 mouse, researchers passage the tumor over to the next generation of mice. Each generation thereafter is denoted F1, F2, F3 . . . . Fn. For drug development studies, expansion of mice between the F3 and F10 generation is often utilized to ensure that the PDX has not genetically or histologically diverged from the patient's tumor.[8]

Advantages over Cancer Cell Lines for Predictive Model Validation

Without wishing to be bound by any particular theory, the present disclosure recognizes one or combinations of several factors that makes PDX mouse models an advantageous tool in comparison to the cancer cell line screening or cancer cell line-derived xenograft models (CDX). First, cancer cell lines are originally derived from patient tumors, but acquire the ability to proliferate within in vitro cell cultures. As a result of in vitro manipulation, cell lines that have been traditionally used in cancer research undergo genetic transformations that are not restored when cells are allowed to grow in vivo. Because of the cell culturing process, which includes enzymatic environments and centrifugation, cells that are better adapted to survive in culture are selected, tumor resident cells and proteins that interact with cancer cells are eliminated, and the culture becomes phenotypically homogeneous.

Second, when implanted into immunodeficient mice, cell lines do not easily develop tumors and the result of any successfully grown tumor is a genetically divergent tumor unlike the heterogeneous patient tumor. Researchers are beginning to attribute the reason that only 5% of anti-cancer agents are approved by the Food and Drug Administration after pre-clinical testing to the lack of tumor heterogeneity and the absence of the human stromal microenvironment. Specifically, cell line-xenografts often are not predictive of the drug response in the primary tumors because cell lines do not follow pathways of drug resistance or the effects of the microenvironment on drug response found in human primary tumors.

Third, many PDX models have been successfully established for breast, prostate, colorectal, lung, and many other cancers because there are distinctive advantages when using PDX over cell lines for drug safety and efficacy studies as well as predicting patient tumor response to certain anti-cancer agents. Since PDX can be passaged without in vitro processing steps, PDX models allow the propagation and expansion of patient tumors without significant genetic transformation of tumor cells over multiple murine generations. Within PDX models, patient tumor samples grow in physiologically-relevant tumor microenvironments that mimic the oxygen, nutrient, and hormone levels that are found in the patient's primary tumor site. Furthermore, implanted tumor tissue maintains the genetic and epigenetic abnormalities found in the patient and the xenograft tissue can be excised from the patient to include the surrounding human stroma. As a result, numerous studies have found that PDX models exhibit similar responses to anti-cancer agents as seen in the actual patient who provided the tumor sample.

### Additional Therapeutic Agents

The present disclosure provides for embodiments where additional therapeutic agents may be administered to a lung cancer patient in need thereof. Additional therapeutic agents may include alkylating agents, antibiotics, cytotoxic antibiotics, antimetabolites, biologic response modifiers, histone deacetylase inhibitors, hormonal agents, monoclonal antibodies, protein kinase inhibitors, taxanes, topoisomerase inhibitors, vinca alkaloids, photoactive therapeutic agents, anti-angiogenesis agents, radiosensitizing agents, radionuclides, immunomodulators, cytotoxic agents, or combinations thereof.

Examples of alkylating agents may include, but are not limited to, altretamine, bendamustine, busulfan, carmustine, chlorambucil, cyclophosphamide, dacarbazine, ifosfamide, lomustine, lurbinectedin, mechlorethamine, melphalan, procarbazine, stretozocin, temozolomide, thiotepa, and trabectedin. Alkylating agents may also include platinum coordination complexes, such as carboplatin, cisplatin, or oxaliplatin.

Examples of cytotoxic antibiotics may include, but are not limited to, bleomycin, dactinomycin, daunorubicin, doxorubicin, epirubicin, idarubicin, mitomycin, mitoxantrone, plicamycin, or valrubicin.

Examples of antimetabolites may include, but are not limited to, antifolates, purine analogues, and pyrimidine analogues. Examples of antifolates may include methotrexate, pemetrexed, pralatrexate, or trimetrexate. Examples of purine analogues may include azathioprine, cladribine, fludarabine, mercaptopurine, or thioguanine. Examples of pyrimidine analogues may include azacytidine, capecitabine, cytarabine, decitabine, floxuridine, fluorouracil, gemcitabine, or trifluridine/tipracil.

Examples of biologic response modifiers may include, but are not limited to, aldesleukin (IL-2), denileukin difititox, and interferon gamma.

Examples of histone deacetylase inhibitors may include, but are not limited to, belinostat, panobinostat, romidepsin, and vorinostat.

Examples of hormonal agents may include, but are not limited to, antiandrogens, antiestrogens, gonadotropin releasing hormone analogues, and peptide hormones. Examples of antiandrogens may include abiraterone, apalutamide, bicalutamide, cyproterone, enzalutamide, flutamide, or nilutamide. Examples of antiestrogens may include aromatase inhibitors, anastrozole, exemestane, fulvestrant, letrozole, raloxifene, tamoxifen, or toremifene. Examples of gonadotropin releasing hormone analogues may include degarelix, goserelin, histrelin, leuprolide, relugolix, or triptorelin. Examples of peptide hormones may include lanreotide, octreotide, or pasireotide.

Examples of monoclonal antibodies may include, but are not limited to, alemtuzumab, atezolizumab, avelumab, bevacizumab, blinatumomab, brentuximab, cemiplimab, cetuximab, daratumumab, dinutuximab, dostarlimab, durvalumab, elotuzumab, gemtuzumab, inotuzumab ozogamicin, ipilimumab, mogamulizumab, moxetumomab pasudotox, necitumumab, nivolumab, ofatumumab, olaratumab, panitumumab, pembrolizumab, pertuzumab, ramucirumab, rituximab, teclistamab, tositumomab, trastuzumab, tremelimumab, or fragments thereof.

Examples of protein kinase inhibitors may include, but are not limited to, abemaciclib, acalabrutinib, afatinib, alectinib, alpelisib, axitinib, binimetinib, bortezomib, bosutinib, brigatinib, cabozantinib, carfilzomib, ceritinib, cobimetinib, copanlisib, crizotinib, dabrafenib, dacomitinib, dasatinib, duvelisib, enasidenb, encorafenib, entrectinib, erdafitinib, erlotinib, fedratinib, futibatinib, gefitinib, gilteritinib, glasdegib, ibrutinib, idelalisib, imatinib, infigratinib, ivosidenib, ixazomib, lapatinib, larotrectinib, lenvatinib, lorlatinib, midostaurin, neratinib, nilotinib, niraparib, olaparib, osimertinib, palbociclib, pzopanib, pemigatinib, pexidartinib, ponatinib, regorafenib, ribocicib, recuparib, ruxolitinib, selumetinib, onidegib, sorafentib, sunitinib, talazoparib, trametinib, vandetanib, vemurafenib, vismodegib, and zanubrutinib.

Examples of taxanes may include, but are not limited to, cabazitaxel, docetaxel, and paclitaxel.

Examples of topoisomerase inhibitors may include, but are not limited to, etoposide, irinotecan, teniposide, and topotecan.

Examples of vinca alkaloids may include, but are not limited to, vinblastine, vincristine, and vinorelbine.

### NON-LIMITING EMBODIMENTS

Provided below are certain non-limiting Embodiments of different aspects of the present disclosure.

Embodiment 1. A method of treating or ameliorating the effects of a solid tumor cancer in a subject in need thereof, the method comprising:
administering to the subject an effective amount of (i) a first anti-cancer agent, which is a PI3K-alpha inhibitor or a pharmaceutically acceptable salt thereof and (ii) a second anti-cancer agent, which is an mTOR inhibitor or a pharmaceutically acceptable salt thereof, to treat or ameliorate the effects of the solid tumor cancer.

Embodiment 2. The method of embodiment 1, wherein the PI3K-alpha inhibitor is selected from the group consisting of: Alpelisib, Omipalisib, Gedatolisib, Inavolisib, PF-04691502, and Serabelisib.

Embodiment 3. The method of embodiment 1, wherein the mTOR inhibitor is selected from the group consisting of: Everolimus, AXD-8055, Tacrolimus, and Ridaforolimus.

Embodiment 4. The method of any of the previous embodiments, wherein the subject is a mammal.

Embodiment 5. The method of embodiment 4, wherein the mammal is selected from the group consisting of: humans, primates, farm animals, and domestic animals.

Embodiment 6. The method of embodiment 4, wherein the mammal is a human.

Embodiment 7. The method of any of the previous embodiments, wherein the administration of the first and second anti-cancer agents provides a synergistic effect compared to administration of either anti-cancer agent alone.

Embodiment 8. The method of embodiment 7, wherein the synergistic effect assessed at least in part using a ZIP synergy assessment.

Embodiment 9. The method of embodiment 7, wherein the synergistic effect assessed at least in part using a Bliss synergy assessment.

Embodiment 10. The method of embodiment 7, wherein the synergistic effect assessed at least in part using a Loewe synergy assessment.

Embodiment 11. The method of embodiment 7, wherein the synergistic effect assessed at least in part using an HSA synergy assessment.

Embodiment 12. The method of embodiment 7, wherein the synergistic effect is assessed by taking a maximal synergy among a plurality of synergy assessment scores.

Embodiment 13. The method of embodiment 7, wherein the synergistic effect is assessed by taking an average synergy of a plurality of synergy assessment scores.

Embodiment 14. The method of embodiment 12 or 13, wherein one or more of the plurality of synergy assessment scores are selected from the group consisting of a ZIP synergy score, a Bliss synergy score, a Loewe synergy score, and an HSA synergy score.

Embodiment 15. The method of any of the previous embodiments, wherein the subject has a 10% lower rate of tumor growth 10 days after administration of the effective amount of the first and second anti-cancer agents as compared to a control.

Embodiment 16. The method of any of the previous embodiments, wherein the subject has a 20% lower rate of tumor growth 10 days after administration of the effective amount of the first and second anti-cancer agents as compared to a control.

Embodiment 17. The method of any of the previous embodiments, wherein the subject has a 50% lower rate of tumor growth 10 days after administration of the effective amount of the first and second anti-cancer agents as compared to a control.

Embodiment 18. The method of any of the previous embodiments, wherein the subject has a 10% lower rate of tumor growth 20 days after administration of the effective amount of the first and second anti-cancer agents as compared to a control.

Embodiment 19. The method of any of the previous embodiments, wherein the subject has a 20% lower rate of tumor growth 20 days after administration of the effective amount of the first and second anti-cancer agents as compared to a control.

Embodiment 20. The method of any of the previous embodiments, wherein the subject has a 50% lower rate of tumor growth 20 days after administration of the effective amount of the first and second anti-cancer agents as compared to a control.

Embodiment 21. The method of any of the previous embodiments, wherein the subject has a 10% lower rate of tumor growth 24 days after administration of the effective amount of the first and second anti-cancer agents as compared to a control.

Embodiment 22. The method of any of the previous embodiments, wherein the subject has a 20% lower rate of tumor growth 24 days after administration of the effective amount of the first and second anti-cancer agents as compared to a control.

Embodiment 23. The method of any of the previous embodiments, wherein the subject has a 50% lower rate of tumor growth 24 days after administration of the effective amount of the first and second anti-cancer agents as compared to a control.

Embodiment 24. The method of any of the previous embodiments, further comprising administering at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

Embodiment 25. A pharmaceutical composition for treating or ameliorating the effects of a solid tumor cancer in a subject in need thereof, the pharmaceutical composition comprising:
a pharmaceutically acceptable diluent or carrier, and an effective amount of (i) a first anti-cancer agent, which is a PI3K-alpha inhibitor or a pharmaceutically acceptable salt thereof and (ii) a second anti-cancer agent, which is an mTOR inhibitor or a pharmaceutically acceptable salt thereof, wherein administration of the first and second anti-cancer agents provides a synergistic effect compared to administration of either anti-cancer agent alone.

Embodiment 26. The pharmaceutical composition of any of the previous embodiments, wherein the subject is a mammal.

Embodiment 27. The pharmaceutical composition of embodiment 26, wherein the mammal is selected from the group consisting of: humans, primates, farm animals, and domestic animals.

Embodiment 28. The pharmaceutical composition of embodiment 26, wherein the mammal is a human.

Embodiment 29. The pharmaceutical composition of any of the previous embodiments, wherein the lung cancer is non-small cell lung cancer.

Embodiment 30. The pharmaceutical composition of any of the previous embodiments, wherein the administration of the first and second anti-cancer agents provides a synergistic effect compared to administration of either anti-cancer agent alone.

Embodiment 31. The pharmaceutical composition of embodiment 30, wherein the synergistic effect assessed at least in part using a ZIP synergy assessment.

Embodiment 32. The pharmaceutical composition of embodiment 30, wherein the synergistic effect assessed at least in part using a Bliss synergy assessment.

Embodiment 33. The pharmaceutical composition of embodiment 30, wherein the synergistic effect assessed at least in part using a Loewe synergy assessment.

Embodiment 34. The pharmaceutical composition of embodiment 30, wherein the synergistic effect assessed at least in part using an HSA synergy assessment.

Embodiment 35. The pharmaceutical composition of embodiment 30, wherein the synergistic effect is assessed by taking a maximal synergy among a plurality of synergy assessment scores.

Embodiment 36. The pharmaceutical composition of embodiment 30, wherein the synergistic effect is assessed by averaging a plurality of synergy assessment scores.

Embodiment 37. The pharmaceutical composition of embodiment 35 or 36, wherein one or more of the plurality of synergy assessment scores are selected from the group consisting of a ZIP synergy score, a Bliss synergy score, a Loewe synergy score, and an HSA synergy score.

Embodiment 38. The pharmaceutical composition of any of the previous embodiments, wherein the subject has a 10% lower rate of tumor growth 10 days after administration of the effective amount of the first and second anti-cancer agents as compared to a control.

Embodiment 39. The pharmaceutical composition of any of the previous embodiments, wherein the subject has a 20% lower rate of tumor growth 10 days after administration of the effective amount of the first and second anti-cancer agents as compared to a control.

Embodiment 40. The pharmaceutical composition of any of the previous embodiments, wherein the subject has a 50% lower rate of tumor growth 10 days after administration of the effective amount of the first and second anti-cancer agents as compared to a control.

Embodiment 41. The pharmaceutical composition of any of the previous embodiments, wherein the subject has a 10% lower rate of tumor growth 20 days after administration of the effective amount of the first and second anti-cancer agents as compared to a control.

Embodiment 42. The pharmaceutical composition of any of the previous embodiments, wherein the subject has a 20% lower rate of tumor growth 20 days after administration of the effective amount of the first and second anti-cancer agents as compared to a control.

Embodiment 43. The pharmaceutical composition of any of the previous embodiments, wherein the subject has a 50% lower rate of tumor growth 20 days after administration of the effective amount of the first and second anti-cancer agents as compared to a control.

Embodiment 44. The pharmaceutical composition of any of the previous embodiments, wherein the subject has a 10% lower rate of tumor growth 24 days after administration of the effective amount of the first and second anti-cancer agents as compared to a control.

Embodiment 45. The pharmaceutical composition of any of the previous embodiments, wherein the subject has a 20% lower rate of tumor growth 24 days after administration of the effective amount of the first and second anti-cancer agents as compared to a control.

Embodiment 46. The pharmaceutical composition of any of the previous embodiments, wherein the subject has a 50% lower rate of tumor growth 24 days after administration of the effective amount of the first and second anti-cancer agents as compared to a control.

Embodiment 47. The pharmaceutical composition of any of the previous embodiments, further comprising at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

Embodiment 48. The pharmaceutical composition of any of the previous embodiments, wherein the pharmaceutical composition is in a unit dosage form comprising both the first and second anti-cancer agents.

Embodiment 49. The pharmaceutical composition of any of the previous embodiments, wherein the first anti-cancer agent is in a first unit dosage form and the second anti-cancer agent is in a second unit dosage form, wherein the first unit dosage form is separate from the second unit dosage form.

Embodiment 50. The pharmaceutical composition of any of the previous embodiments, wherein the first and second anti-cancer agents are co-administered to the subject.

Embodiment 51. The pharmaceutical composition of any of the previous embodiments, wherein the first and second anti-cancer agents are administered to the subject serially.

Embodiment 52. The pharmaceutical composition of embodiment 50, wherein the first anti-cancer agent is administered to the subject before the second anti-cancer agent.

Embodiment 53. The pharmaceutical composition of embodiment 50, wherein the second anti-cancer agent is administered to the subject before the first anti-cancer agent.

Embodiment 54. A data structure comprising a decision tree random forest model that is utilized to uncover biomarkers to predict the efficacy of a PI3K-alpha inhibitor or a pharmaceutically acceptable salt thereof and a second anti-cancer agent, wherein the second anti-cancer agent is an mTOR inhibitor or a pharmaceutically acceptable salt thereof.

Embodiment 55. A method of predicting the response to treating or ameliorating the effects of a solid tumor cancer of embodiment 1 using measurements of gene expression biomarkers of four or more genes selected from the group consisting of: RPS16, HERC6, L2HGDH, AAGAB, CENPN, CHMP2B, TMED10.

Embodiment 56. The method of embodiment 55, wherein the method of predicting further uses assessments of the ratio of the predictive biomarkers of the one or more gene expression biomarker measurements as compared to a geometric mean of measurements of one or more housekeeping genes selected from the group consisting of: RPL27, RPL4, UBA52, HNRNPK, RPL30.

Embodiment 57. A method of predicting the response to treating or ameliorating the effects of a solid tumor cancer of embodiment 1 using a molecular description of a PI3K-alpha inhibitor or a pharmaceutically acceptable salt thereof.

Embodiment 58. A method of predicting the response to treating or ameliorating the effects of a solid tumor cancer of embodiment 1 using a molecular description of an mTOR inhibitor or a pharmaceutically acceptable salt thereof.

Embodiment 59. A method of predicting the response to treating or ameliorating the effects of a solid tumor cancer of embodiment 1 using a molecular fingerprint of a PI3K-alpha inhibitor or a pharmaceutically acceptable salt thereof.

Embodiment 60. A method of predicting the response to treating or ameliorating the effects of a solid tumor cancer of embodiment 1 using a molecular fingerprint of an mTOR inhibitor or a pharmaceutically acceptable salt thereof.

### NON-LIMITING EXAMPLES

Provided below are certain non-limiting examples according to different aspects of the present disclosure.

### Example I—PI3K-Alpha Inhibitors and mTOR Inhibitors

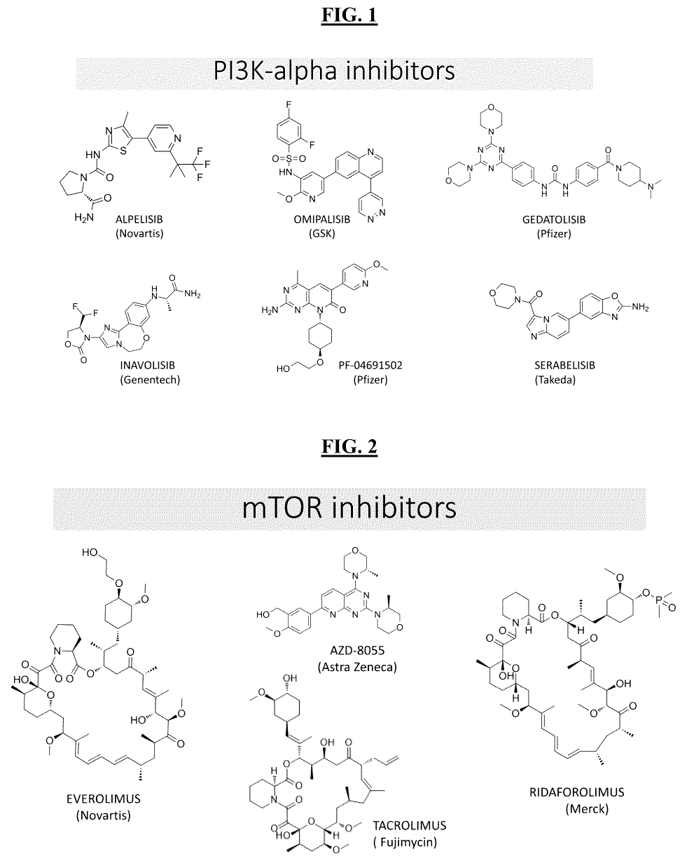

FIG. 1 provides an illustration of a plurality of PI3K-alpha inhibitor molecules, including Alpelisib, Omipalisib, Gedatolisib, Inavolisib, PF-04691502, and Serabelisib.

FIG. 2 provides an illustration of a plurality of mTOR inhibitor molecules, including Everolimus, AXD-8055, Tacrolimus, and Ridaforolimus.

### Example II—In Vitro Synergy of Drug Combinations

Table 1 provides in-vitro synergy results of mTOR and PI3K combinations in various solid tumor PDX-derived cell lines. Synergy is calculated for each pairwise combination of single-drug concentration mixtures. The max synergy score is derived from the highest synergy score of the four synergy calculation models (ZIP, HSA, Bliss, and Loewe).

In particular, Table 1 illustrates categories of “Model,” “CancerType,” “TimePoint,” “Drug1,” “Drug2,” “ZIP,” HAS,” “Bliss,” Loewe,” and “MaxSynergy.” Table 1 also is presented in this document across two portions: a first portion on the first page, and a continued portion on a second page.

TABLE 1

| Model | CancerType | Drug1 | Drug2 | ZIP | HSA | Bliss | Loewe | MaxSynergy |
|---|---|---|---|---|---|---|---|---|
| CRT00292 | Gastric | Everolimus | Alpelisib | 6.321327 | 9.802703 | 4.257724 | 11.40621 | 11.40620924 |
| CRT00295 | Lung | Everolimus | Alpelisib | 12.52011 | 0.252273 | 9.470765 | −0.17551 | 12.52011065 |
| CRT00295 | Lung | Alpelisib | Ridaforolimus | 11.5794 | −3.75412 | 6.369562 | −3.66365 | 11.57940241 |
| CRT00295 | Lung | Alpelisib | AZD8055 | 10.21234 | −4.82261 | 5.477911 | −5.13555 | 10.2123405 |
| CRT00295 | Lung | Inavolisib | Tacrolimus | 10.20204 | −1.00715 | 6.647229 | 0.786136 | 10.20203904 |
| CRT00295 | Lung | Everolimus | Inavolisib | 6.450273 | 5.702773 | 3.305799 | 6.379678 | 6.450273126 |
| CRT00295 | Lung | Everolimus | Gedatosilib | 3.186814 | 4.683114 | 1.408137 | 2.606838 | 4.683114248 |
| CRT00295 | Lung | Serabelisib | Ridaforolimus | 3.131462 | −7.19943 | −4.02026 | −6.14572 | 3.131461965 |
| CRT00295 | Lung | Inavolisib | Ridaforolimus | −0.06773 | 2.641579 | −1.48599 | 1.390674 | 2.641578981 |
| CRT00295 | Lung | Alpelisib | Tacrolimus | 2.27943 | −11.208 | −3.32521 | −11.9858 | 2.279430296 |
| CRT00295 | Lung | Everolimus | PF-04691502 | −2.93321 | 1.238172 | −4.8238 | 1.722834 | 1.72283385 |
| CRT00295 | Lung | Serabelisib | Tacrolimus | 1.211671 | −4.58709 | −0.04745 | −4.38734 | 1.211670791 |
| CRT00295 | Lung | Everolimus | Serabelisib | 0.90646 | −3.71392 | −5.06027 | −2.71837 | 0.90645981 |
| CRT00295 | Lung | PF-04691502 | Tacrolimus | −0.42149 | −4.35266 | −4.72888 | −4.34093 | −0.421487729 |
| CRT00295 | Lung | Inavolisib | AZD8055 | −0.5321 | −0.76199 | −1.88146 | −0.72147 | −0.532101938 |
| CRT00295 | Lung | Everolimus | Omipasilib | −5.94159 | −0.76909 | −8.55761 | −1.47259 | −0.76908792 |
| CRT00295 | Lung | Omipasilib | Ridaforolimus | −8.80522 | −1.53265 | −11.5459 | −1.85264 | −1.532647841 |
| CRT00295 | Lung | PF-04691502 | AZD8055 | −2.0711 | −2.99086 | −5.50927 | −3.61643 | −2.071102994 |
| CRT00295 | Lung | PF-04691502 | Ridaforolimus | −8.43287 | −2.86916 | −12.4264 | −2.83329 | −2.833290337 |
| CRT00295 | Lung | Omipasilib | Tacrolimus | −2.8834 | −3.92295 | −6.01157 | −15.5738 | −2.883396704 |
| CRT00295 | Lung | Serabelisib | AZD8055 | −4.59851 | −8.96989 | −10.3428 | −9.3951 | −4.598505627 |
| CRT00295 | Lung | Omipasilib | AZD8055 | −10.3137 | −5.47625 | −13.7383 | −5.86123 | −5.476246617 |
| CRT00295 | Lung | Gedatolisib | Tacrolimus | −5.76361 | −9.70604 | −12.8014 | −10.0971 | −5.763613507 |
| CRT00295 | Lung | Gedatolisib | Ridaforolimus | −10.5627 | −5.97435 | −16.9476 | −5.76485 | −5.76485037 |
| CRT00295 | Lung | Gedatolisib | AZD8055 | −10.8192 | −5.84801 | −14.8644 | −6.14503 | −5.848008118 |
| CRT00354 | Pancreas | Everolimus | Alpelisib | −7.20708 | −6.95973 | −12.9589 | −4.2336 | −4.233602855 |
| CRT00400 | Pancreas | Everolimus | Alpelisib | 3.071426 | −8.59356 | −4.88621 | −7.5692 | 3.071426469 |
| CRT00409 | Pancreas | Everolimus | Alpelisib | 4.989558 | 4.795403 | 2.06533 | 5.445573 | 5.445572838 |
| CRT00419 | Pancreas | Everolimus | Alpelisib | −3.97635 | −3.86826 | −7.81532 | −3.58352 | −3.583519658 |
| CRT00516 | Colon | Everolimus | Alpelisib | 7.563921 | −0.04706 | 3.886855 | 2.02721 | 7.56392103 |
| CRT00559 | Pancreas | Everolimus | Alpelisib | −0.51143 | −10.4459 | −5.65283 | −8.87837 | −0.511427241 |
| CRT00647 | Pancreas | Everolimus | Alpelisib | −11.4728 | −4.91291 | −13.9267 | −5.58939 | −4.912906372 |
| CRT00648 | Pancreas | Everolimus | Alpelisib | 3.103736 | −6.85809 | −0.74414 | −6.67479 | 3.103736287 |
| CRT00673 | Lung | Everolimus | Alpelisib | −7.85013 | −1.62822 | −10.39 | −2.01602 | −1.628218858 |
| CRT00673 | Lung | Tacrolimus | Inavolisib | −15.8351 | −15.7067 | −22.9024 | −15.2739 | −15.27389459 |
| CRT00687 | Lung | Everolimus | Alpelisib | 7.793469 | 1.229307 | 8.155715 | 0.509817 | 8.155715498 |
| CRT00705 | Lung | Everolimus | Alpelisib | 1.613016 | −1.70689 | 3.783438 | −2.25244 | 3.7834376 |

Table 2 provides in-vitro Area Under the Response Curve (AUC) calculations of mTOR and PI3K combinations in various solid tumor derived cell lines.

TABLE 2

| Model | CancerType | Timepoint | Drug1 | Drug2 | AUC Avg |
|---|---|---|---|---|---|
| CRT00292 | Gastric | Day 4 | Everolimus | Alpelisib | 0.51717827 |
| CRT00295 | Lung | Day 4 | Alpelisib | AZD8055 | 0.70095073 |
| CRT00295 | Lung | Day 4 | Alpelisib | Deforolimus | 0.74251004 |
| CRT00295 | Lung | Day 4 | Alpelisib | Tacrolimus | 0.84578647 |
| CRT00295 | Lung | Day 4 | Everolimus | Alpelisib | 0.78875938 |
| CRT00295 | Lung | Day 4 | Everolimus | Gedatolisib | 0.43865542 |
| CRT00295 | Lung | Day 4 | Everolimus | Inavolisib | 0.48666242 |
| CRT00295 | Lung | Day 4 | Everolimus | Omipalisib | 0.30903798 |
| CRT00295 | Lung | Day 4 | Everolimus | PF-04691502 | 0.32294397 |
| CRT00295 | Lung | Day 4 | Everolimus | Serabelisib | 0.50514007 |
| CRT00295 | Lung | Day 4 | Gedatolisib | AZD8055 | 0.13145167 |
| CRT00295 | Lung | Day 4 | Gedatolisib | Deforolimus | 0.18535601 |
| CRT00295 | Lung | Day 4 | Gedatolisib | Tacrolimus | 0.2764574 |
| CRT00295 | Lung | Day 4 | Inavolisib | AZD8055 | 0.48137495 |
| CRT00295 | Lung | Day 4 | Inavolisib | Deforolimus | 0.51349279 |
| CRT00295 | Lung | Day 4 | Inavolisib | Tacrolimus | 0.89094356 |
| CRT00295 | Lung | Day 4 | Omipalisib | AZD8055 | 0.11836518 |
| CRT00295 | Lung | Day 4 | Omipalisib | Deforolimus | 0.20783264 |
| CRT00295 | Lung | Day 4 | Omipalisib | Tacrolimus | 0.23470681 |
| CRT00295 | Lung | Day 4 | PF-04691502 | AZD8055 | 0.19787061 |
| CRT00295 | Lung | Day 4 | PF-04691502 | Deforolimus | 0.20586839 |
| CRT00295 | Lung | Day 4 | PF-04691502 | Tacrolimus | 0.31002167 |
| CRT00295 | Lung | Day 4 | Serabelisib | AZD8055 | 0.19790729 |
| CRT00295 | Lung | Day 4 | Serabelisib | Deforolimus | 0.62499756 |
| CRT00295 | Lung | Day 4 | Serabelisib | Tacrolimus | 0.55904172 |
| CRT00354 | Pancreas | Day 4 | Everolimus | Alpelisib | 0.42795928 |
| CRT00400 | Pancreas | Day 4 | Everolimus | Alpelisib | 0.77810762 |
| CRT00409 | Pancreas | Day 4 | Everolimus | Alpelisib | 0.69534905 |
| CRT00419 | Pancreas | Day 4 | Everolimus | Alpelisib | 0.46477022 |
| CRT00516 | Colon | Day 4 | Everolimus | Alpelisib | 0.70034648 |
| CRT00559 | Pancreas | Day 4 | Everolimus | Alpelisib | 0.81624659 |
| CRT00647 | Pancreas | Day 4 | Everolimus | Alpelisib | 0.39455579 |
| CRT00648 | Pancreas | Day 4 | Everolimus | Alpelisib | 0.78160607 |
| CRT00673 | Lung | Day 4 | Everolimus | Alpelisib | 0.52049551 |
| CRT00673 | Lung | Day 4 | Tacrolimus | Inavolisib | 0.1856531 |
| CRT00687 | Lung | Day 4 | Everolimus | Alpelisib | 0.50915539 |
| CRT00705 | Lung | Day 4 | Everolimus | Alpelisib | 0.7022072 |
| CRT00705 | Lung | Day 4 | Everolimus | Alpelisib | 0.91718127 |

Table 3 provides analysis results from a random forest regression model trained on the 44 in-vitro experiments. Both the Max Synergy from table 1 and AUC from table 2 were used as endpoints for training.

TABLE 3
MTOR-PI3K Random Forest Regression Model

| | |
|---|---|
| Training Accuracy: | 0.9216 |
| Testing Score: | 0.6082 |
| RMSE (AUC): | 0.1743 |

### Example III—In Vivo Pharmacology of Drug Combinations

Table 4 provides in-vivo results of Everolimus and Alpelisib combination in Patient Derived Xenograft (PDX) solid tumor models as well as predicted response from the random forest regression model.

TABLE 4

| | | | Actual | Predicted |
|---|---|---|---|---|
| Model | CancerType | Treatment | TGI | TGI |
| CRT00292 | Gastric | Everolimus + | 87.1556 | 104.2608 |
| | | Alpelisib | | |
| CRT00295 | Lung | Everolimus + | 114.4267 | 106.6238 |
| | | Alpelisib | | |
| CRT00316 | Sarcoma | Everolimus + | 78.6188 | 106.9176 |
| | | Alpelisib | | |
| CRT00516 | Colorectal | Everolimus + | 70.2608 | 82.0381 |
| | | Alpelisib | | |
| CRT00538 | Breast | Everolimus + | 80.4278 | 98.7915 |
| | | Alpelisib | | |
| CRT00554 | Colorectal | Everolimus + | 60.2500 | 97.7007 |
| | | Alpelisib | | |
| CRT00559 | Pancreatic | Everolimus + | 58.0520 | 74.1884 |
| | | Alpelisib | | |
| CRT00581 | Colorectal | Everolimus + | 60.7597 | 89.0445 |
| | | Alpelisib | | |
| CRT00683 | Lung | Everolimus + | 82.2553 | 99.2839 |
| | | Alpelisib | | |
| CRT00687 | Lung | Everolimus + | 73.6687 | 110.1642 |
| | | Alpelisib | | |
| CRT00731 | Sarcoma | Everolimus + | 95.5712 | 103.4224 |
| | | Alpelisib | | |

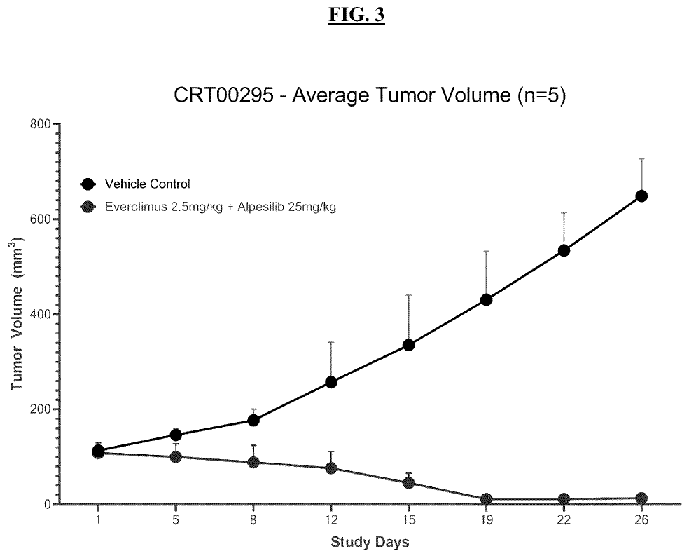

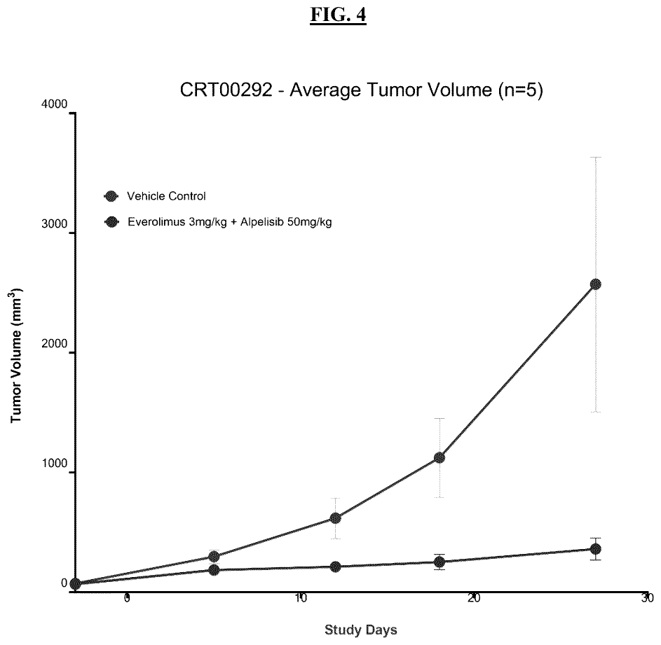

FIG. 3 illustrates in-vivo pharmacology test results of a Patient Derived Xenograft (PDX) lung cancer model, CRT00295. On the x-axis, tumor volume is represented, and on the y-axis, study days is represented. Further, two sets of data are plotted: a first set of data provides a PDX model of a mouse that is a vehicle control, while a second set of data provides a PDX model of a mouse treated with a combination of Everolimus and Alpelisib.

FIG. 4 illustrates in-vivo pharmacology test results of a Patient Derived Xenograft (PDX) gastric cancer model, CRT00292. On the x-axis, tumor volume is represented, and on the y-axis, study days is represented. Further, two sets of data are plotted: a first set of data provides a PDX model of a mouse that is a vehicle control, while a second set of data provides a PDX model of a mouse treated with a combination of Everolimus and Alpelisib.

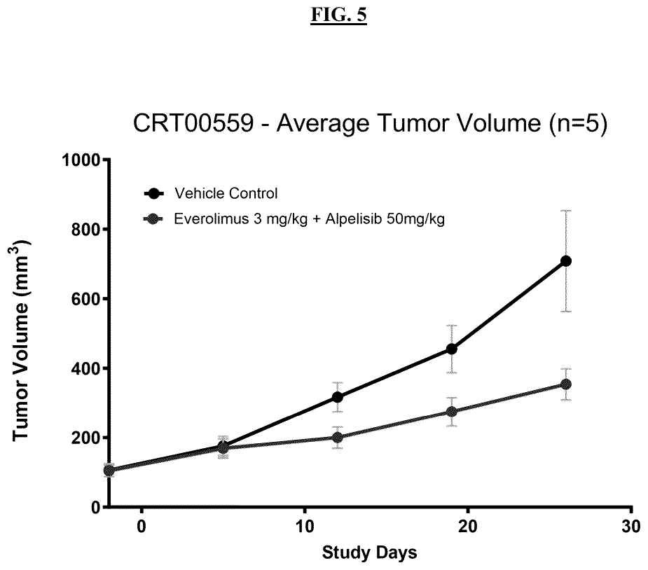

FIG. 5 illustrates in-vivo pharmacology test results of a Patient Derived Xenograft (PDX) pancreatic cancer model, CRT00559. On the x-axis, tumor volume is represented, and on the y-axis, study days is represented. Further, two sets of data are plotted: a first set of data provides a PDX model of a mouse that is a vehicle control, while a second set of data provides a PDX model of a mouse treated with a combination of Everolimus and Alpelisib.

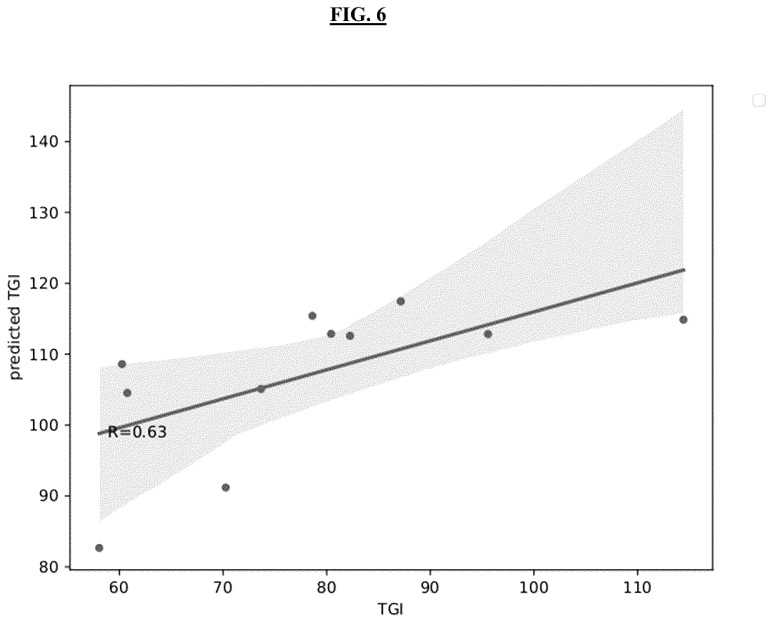

FIG. 6 illustrates a comprehensive correlation analysis between predicted tumor growth inhibition (TGI) vs. actual TGI from in-vivo Everoliumus and Alpelisib combination pharmacology studies across 11 PDX solid tumor models (Table 4) showing a positive Pearson's correlation of r=0.63.

What is claimed is:

1. A method of treating or ameliorating the effects of a solid tumor cancer in a subject in need thereof, the method comprising:
administering to the subject an effective amount of (i) a first anti-cancer agent, which is Inavolisib or a pharmaceutically acceptable salt thereof and (ii) a second anti-cancer agent selected from Tacrolimus or Everolimus, or a pharmaceutically acceptable salt thereof, to treat or ameliorate the effects of the solid tumor cancer.

2. The method of claim 1, wherein the second anti-cancer agent is Tacrolimus.

3. The method of claim 1, the second anti-cancer agent is Everolimus.

4. The method of claim 1, wherein the subject has a 10% lower rate or less of tumor growth 10 days after administration of the effective amount of the first and second anti-cancer agents as compared to tumor growth before administration of the effective amount of the first and second anti-cancer agents.

5. The method of claim 1, further comprising administering at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

6. The method of claim 1, wherein the subject is a mammal.

7. The method of claim 6, herein the mammal is selected from the group consisting of: humans, primates, farm animals, and domestic animals.

8. The method of claim 6, wherein the mammal is a human.

9. The method of claim 1, wherein the administration of the first and second anti-cancer agents provides a synergistic effect compared to administration of either anti-cancer agent alone.

10. The method of claim 9, wherein the synergistic effect is assessed at least in part using a ZIP synergy assessment.

11. The method of claim 9, wherein the synergistic effect is assessed at least in part using a Bliss synergy assessment.

12. The method of claim 9, wherein the synergistic effect is assessed at least in part using a Loewe synergy assessment.

13. The method of claim 9, wherein the synergistic effect is assessed at least in part using an HSA synergy assessment.

14. The method of claim 9, wherein the synergistic effect is assessed by taking a maximal synergy among a plurality of synergy assessment scores.

15. The method of claim 9, wherein the synergistic effect is assessed by taking an average synergy of a plurality of synergy assessment scores.

16. The method of claim 14, wherein one or more of the plurality of synergy assessment scores are selected from the group consisting of a ZIP synergy score, a Bliss synergy score, a Loewe synergy score, and an HSA synergy score.

17. The method of claim 15, wherein one or more of the plurality of synergy assessment scores are selected from the group consisting of a ZIP synergy score, a Bliss synergy score, a Loewe synergy score, and an HSA synergy score.

18. The method of claim 1, wherein the first anti-cancer agent is in a first unit dosage form and the second anti-cancer agent is in a second unit dosage form, wherein the first unit dosage form is separate from the second unit dosage form.

19. The method of claim 1, wherein the first and second anti-cancer agents are co-administered to the subject.

20. The method of claim 1, wherein the first and second anti-cancer agents are administered to the subject serially.
